(19) United States
(12) Patent Application Publication
(10) Pub. No.: US 2026/0278974 A1
(43) Pub. Date: Sep. 17, 2026

(54) IMAGING METHOD AND MEANS FOR COGNITIVE BRAIN SCIENCE BASED ON METAVERSE MIXED REALITY TECHNOLOGY

(71) Applicant: Nanjing Drum Tower Hospital, Affiliated Hospital of Medical School, Nanjing University, Nanjing (CN)

(72) Inventors: Guan LI, Nanjing (CN); Bing ZHANG, Nanjing (CN); Xin ZHANG, Nanjing (CN); Jun CHEN, Nanjing (CN)

(73) Assignee: Nanjing Drum Tower Hospital, Affiliated Hospital of Medical School, Nanjing University, Nanjing (CN)

(21) Appl. No.: 19/415,862

(22) Filed: Dec. 11, 2025

(30) Foreign Application Priority Data
Mar. 13, 2025 (CN) 2025102994043

(51) Int. Cl. G06T 19/20; A61B 5/00; A61B 5/055; G06T 5/50; G06T 5/70; G06T 7/00; G09B 9/00; G16H 50/50

(52) U.S. Cl. CPC G06T 19/20; A61B 5/0042; A61B 5/055; A61B 5/4088; A61B 5/4842; G06T 5/50; G06T 5/70; G06T 7/0012; G09B 9/00; G16H 50/50; G06T 2207/10088; G06T 2207/20076; G06T 2207/30016; G06T 2207/30096; G06T 2210/32; G06T 2210/41; G06T 2219/2012

(57) ABSTRACT

An imaging method and means for cognitive brain science based on metaverse mixed reality technology are provided, which can provide a clear, intuitive, and highly interactive brain function teaching platform, enhancing the efficiency and immersion of medical education, neuroscience research, and clinical training. The method includes the following steps: (1) modeling brain tissue based on MRI images; (2) virtual modeling fiber bundles; (3) optimizing the model; (4) converting and generating a model.

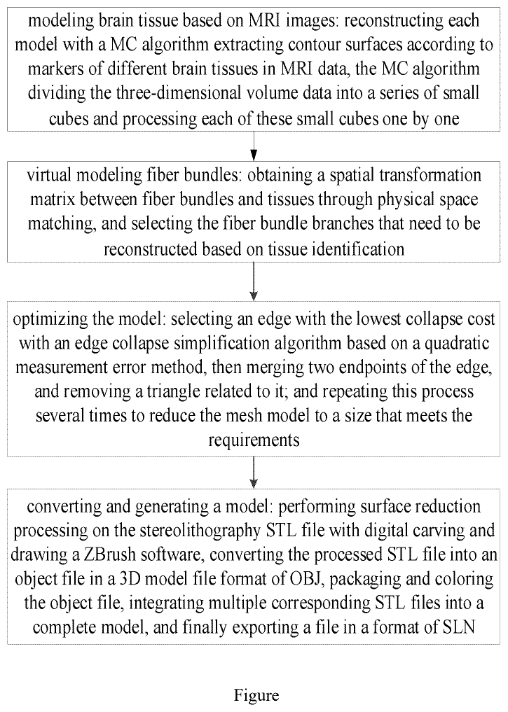

### CROSS-REFERENCE TO THE RELATED APPLICATIONS

This application is based upon and claims priority to Chinese Patent Application No. 202510299404.3, filed on Mar. 13, 2025, the entire contents of which are incorporated herein by reference.

### TECHNICAL FIELD

The present invention relates to the technical fields of biomedical and virtual simulation, particularly to an imaging method for cognitive brain science based on metaverse mixed reality technology, and an imaging means for cognitive brain science based on metaverse mixed reality technology.

### BACKGROUND

A brain is the most complex organ in a human body, with its structure, function, and interrelationships far more complex than any other organ. The research on brain tissue and function in the medical field faces numerous challenges, especially in teaching. How to accurately, comprehensively, and effectively impart knowledge and skills related to the brain is a highly complex and constantly evolving task. Virtual simulation technology is to simulate real-life scenarios with computer-generated virtual worlds. The combination of virtual simulation technology and medical imaging can display three-dimensional images of various parts, tissues, organs, and disease lesions of the human body, enhancing learners' understanding and application of medical knowledge.

In the past, traditional textbook maps and imaging films used two-dimensional displays of brain functional areas, cognitive areas in brain science, neural pathways, and brain anatomy structures, which made it difficult for a user to achieve a three-dimensional sense of space and understanding and cognition of abstract regions. And the user cannot actually operate it. In recent years, 3D printing technology has been commonly used for objective simulation and display of brain tissue. However, 3D printing technology has disadvantages such as long-time consumption, high cost, and poor flexibility, as well as spatial constraints.

### SUMMARY

In order to overcome the defects of the prior art, the technical problem to be solved by the present invention is to provide an imaging method for cognitive brain science based on metaverse mixed reality technology, which can provide a clear, intuitive, and highly interactive brain function teaching platform, enhancing the efficiency and immersion of medical education, neuroscience research, and clinical training.

The technical scheme of the invention is as follows.

The imaging method for cognitive brain science based on metaverse mixed reality technology, including the following steps:
(1) modeling brain tissue based on magnetic resonance imaging (MRI) images: reconstructing each model with a marching cubes (MC) algorithm extracting contour surfaces according to markers of different brain tissues in MRI data, the MC algorithm dividing the three-dimensional volume data into a series of small cubes and processing each of these small cubes one by one;
(2) virtual modeling fiber bundles: obtaining a spatial transformation matrix between fiber bundles and tissues through physical space matching, and selecting the fiber bundle branches that need to be reconstructed based on tissue identification;
(3) optimizing the model: selecting an edge with the lowest collapse cost with an edge collapse simplification algorithm based on a quadratic measurement error method, then merging two endpoints of the edge, and removing a triangle related to it; and repeating this process several times to reduce the mesh model to a size that meets the requirements;
(4) converting and generating a model: performing surface reduction processing on the stereolithography STL file with digital carving and drawing a ZBrush software, converting the processed STL file into an object file in a 3D model file format of OBJ, packaging and coloring the object file, integrating multiple corresponding STL files into a complete model, and finally exporting a file in a format of SLN.

In the present invention an advanced 3D modeling technology is adopted to mark brain structural zones and construct an accurate 3D mesh model. Brain nerve fiber bundles are calculated by processing CT and MRI image data and converted into the three-dimensional mesh model, achieving a high degree of simulation of brain structure and nerve fiber bundles. The model is rendered with a mixed reality device. Users can interact in a virtual and real environment, real-time view the structure and function of various regions of the brain, and the development and changes of cognitive impairment. Color changes are used to simulate the activation of functional areas in different regions of the brain and the transmission process of neural signals, thus presenting the dynamic working state of the brain under different tasks. Therefore, a clear, intuitive and highly interactive brain function teaching platform is provided, which enhances the efficiency and immersion of medical education, neuroscience research and clinical training.

An imaging means for cognitive brain science based on metaverse mixed reality technology is also provided, including:
\textcircled{1} a model of modeling brain tissues based on MRI images, which is configured to reconstruct each model with an MC algorithm extracting contour surfaces according to markers of different brain tissues in MRI data, the MC algorithm dividing the three-dimensional volume data into a series of small cubes and processing each of these small cubes one by one;
\textcircled{2} a model of virtual modeling fiber bundles, which is configured to obtain a spatial transformation matrix between fiber bundles and tissues through physical space matching, and select the fiber bundle branches that need to be reconstructed based on tissue identification;
\textcircled{3} a model of optimizing the model, which is configured to select an edge with the lowest collapse cost with an edge collapse simplification algorithm based on a quadratic measurement error method, then merge two endpoints of the edge, and remove a triangle related to it; and repeat this process several times to reduce the mesh model to a size that meets the requirements;
\textcircled{4} a module of converting and generating a model, which is configured to perform surface reduction processing on the stereolithography STL file with, convert the processed STL file into an object file in a 3D model file format of OBJ, package and color the object file, integrate multiple corresponding STL files into a complete model, and finally export a file in a format of SLN.

### BRIEF DESCRIPTION OF THE DRAWINGS

FIGURE shows a flowchart of the imaging method for cognitive brain science based on metaverse mixed reality technology according to the invention.

### DETAILED DESCRIPTION OF THE EMBODIMENTS

As shown as FIGURE, the imaging method for cognitive brain science based on metaverse mixed reality technology, including the following steps:
(1) modeling brain tissue based on MRI images: reconstructing each model with an MC algorithm extracting contour surfaces according to markers of different brain tissues in MRI data, the MC algorithm dividing the three-dimensional volume data into a series of small cubes and processing each of these small cubes one by one;
(2) virtual modeling fiber bundles: obtaining a spatial transformation matrix between fiber bundles and tissues through physical space matching, and selecting the fiber bundle branches that need to be reconstructed based on tissue identification;
(3) optimizing the model: selecting an edge with the lowest collapse cost with an edge collapse simplification algorithm based on a quadratic measurement error method, then merging two endpoints of the edge, and removing a triangle related to it; and repeating this process several times to reduce the mesh model to a size that meets the requirements;
(4) converting and generating a model: performing surface reduction processing on the stereolithography STL file with digital carving and drawing a ZBrush software, converting the processed STL file into an object file in a 3D model file format of OBJ, packaging and coloring the object file, integrating multiple corresponding STL files into a complete model, and finally exporting a file in a format of SLN.

In the present invention an advanced 3D modeling technology is adopted to mark brain structural zones and construct an accurate 3D mesh model. Brain nerve fiber bundles are calculated by processing CT and MRI image data and converted into the three-dimensional mesh model, achieving a high degree of simulation of brain structure and nerve fiber bundles. The model is rendered with a mixed reality device. Users can interact in a virtual and real environment, real-time view the structure and function of various regions of the brain, and the development and changes of cognitive impairment. Color changes are used to simulate the activation of functional areas in different regions of the brain and the transmission process of neural signals, thus presenting the dynamic working state of the brain under different tasks. Therefore, a clear, intuitive and highly interactive brain function teaching platform is provided, which enhances the efficiency and immersion of medical education, neuroscience research and clinical training.

Preferably, in step (1), the input overall 3D volume data are divided into a series of small cubes, and vertex states of each cube are analyzed one by one. A binary index is generated to query a predefined lookup table and determine corresponding triangular facets with the relationship between marker values of the 8 vertices of the small cube and the preset threshold. A relatively smooth surface is constructed on a cube boundary to complete the reconstruction of the 3D model by inserting equivalent points on the corresponding edge with a linear interpolation method.

Preferably, step (2) including the following sub steps:

(2.1) calculating a direction vector of each point in the fiber bundles, for a certain point in the fiber bundles pᵢ, its direction vector dᵢ is:

$${d}_{i} = {p}_{i + 1} - {p}_{i}$$

(2.2) normalizing the direction vector of the point and converting it into color information:

$${d}_{i}^{′} = \frac{\vert {d}_{i} \vert}{max ( \vert {d}_{i} \vert ) + ϵ} \cdot 255$$

in which E is a small constant to prevent zero division, dᵢ is an absolute value of each component of the direction vector;

(2.3) each nerve bundle is represented by a series of continuous three-dimensional coordinate points; firstly, calculating a tangent vector T_{i,i+1} of each point on the nerve bundle path, which describes a local direction of the path and generates a rotation axis of a cylindrical cross-section; meanwhile, calculating a normal Nᵢ of each point, which provides a normal vector for the generated circular cross-section; for each nerve bundle point, generate a cylindrical cross-section around it, and calculating the positions of each vertex generated on the cross-section as follows:

$$P ( t ) = {P}_{i} + {r}_{i} \cdot ( cos ( t ) \cdot {N}_{i} + sin ( t ) \cdot {T}_{i , i + 1} )$$

in which Pᵢ is a coordinate of a current point in the nerve bundle; rᵢ is a pipe radius of this point and is preset according to the characteristics of the nerve bundle; t is an angle parameter on the circumference with a range of [0, 2π] to generate a complete cylindrical cross-section; the generated cylindrical cross-section connects each pair of adjacent points along the nerve bundle path, forming a complete cylinder. These cylindrical cross-sections are connected one by one along the path direction to construct a continuous pipe. The connection points of these cylindrical segments are calculated with a linear interpolation method and smooth transition of normal vectors to ensure a smooth transition on the pipe surface.

Preferably, in step (3), further including: performing Laplace smoothing on the mesh model, and moving the coordinate of each vertex according to a mean value of the coordinates of its surrounding vertices.

Preferably, in step (4), after obtaining the file in a format of SLN, opening it with Visual Studio 2019 and generating a program package to obtain a file in a format of MSIX.

Preferably, in step (4), importing the file in a format of SLN into a 3D reconstruction workstation to adjust color and transparency.

Preferably, the method also includes step (5), in which uploading the file in a format of MSIX to cloud storage after generating it.

Preferably, the method also includes step (6), in which exporting the data as a file in a format of V3D and generating the corresponding quick response (QR) code with the file in a format of V3D.

Those skilled in the art can understand that all or part of the steps in the method of realizing the above embodiment can be completed by instructing relevant hardware through a program. The program can be stored in a computer-readable storage medium. When the program is executed, it includes the steps of the method of the above embodiment, and the storage medium can be ROM/RAM, magnetic disc, optical disc, memory card, etc. Therefore, corresponding to the method of the invention, the invention also includes an imaging means for cognitive brain science based on metaverse mixed reality technology, which is usually represented in the form of functional modules corresponding to each step of the method. The means includes:
\textcircled{1} a model of modeling brain tissues based on MRI images, which is configured to reconstruct each model with an MC algorithm extracting contour surfaces according to markers of different brain tissues in MRI data, the MC algorithm dividing the three-dimensional volume data into a series of small cubes and processing each of these small cubes one by one;
\textcircled{2} a model of virtual modeling fiber bundles, which is configured to obtain a spatial transformation matrix between fiber bundles and tissues through physical space matching, and select the fiber bundle branches that need to be reconstructed based on tissue identification;
\textcircled{3} a model of optimizing the model, which is configured to select an edge with the lowest collapse cost with an edge collapse simplification algorithm based on a quadratic measurement error method, then merge two endpoints of the edge, and remove a triangle related to it; and repeat this process several times to reduce the mesh model to a size that meets the requirements;
\textcircled{4} a module of converting and generating a model, which is configured to perform surface reduction processing on the stereolithography STL file with, convert the processed STL file into an object file in a 3D model file format of OBJ, package and color the object file, integrate multiple corresponding STL files into a complete model, and finally export a file in a format of SLN.

Preferably, the means further includes a module of uploading, which is configured to upload the file in a format of MSIX to cloud storage after generating it.

Preferably, the means further includes a module of QR code, which is configured to export the data as a file in a format of V3D, and generate the corresponding QR code with the file in a format of V3D or directly input them into a mixed reality glasses HoloLens 2.

The beneficial technical effects of the present invention are as follows.

1. Realize holographic visualization mixed reality imaging of anatomical structures and neural pathways in brain functional areas.

The structure of the brain consists of a frontal lobe, parietal lobe, temporal lobe, occipital lobe, insula lobe, and limbic lobe. The frontal lobe is related to mental, language, and voluntary movement. The parietal lobe can distinguish advanced sensations. The functions of the temporal lobe include language comprehension, hearing, mental activity, memory, smell, etc. The occipital lobe is responsible for processing visual information. The insula lobe is mainly related to visceral sensation and movement. The limbic lobe is involved in advanced neural, mental, emotional, memory, and visceral activities. The imaging of brain functional areas and nerve conduction pathways is achieved by the mixed reality technology, making abstract brain tissue structures vivid and visual, and achieving perspective imaging of nerve conduction disorders and brain tissue lesions, providing a new method for innovative clinical diagnosis and treatment models and teaching guidance.

2. Realize mixed reality imaging of hippocampal evolution in cognitive impairment.

Brain hippocampus, also known as the hippocampus, hippocampus gyrus, or hippocampal region, plays a crucial role in learning, memory, spatial cognition, emotional regulation, and adaptation to the environment. The brain hippocampus is located deep in the temporal lobe of the brain, forming an arc-shaped structure around choroid plexus of the lateral ventricle. It is an important component of the brain. Although not large in size, its function is exceptionally powerful. Damage and diseases of the brain hippocampus can lead to serious cognitive and emotional disorders, with Alzheimer's disease being the most common. AD mainly manifests as symptoms such as memory impairment, cognitive decline, and emotional disorders. Research has shown that atrophy of the hippocampus and loss of neurons are important pathological features of Alzheimer's disease. Changes in the anatomical area and volume of the hippocampus are achieved by mixed reality technology, thus objective observation of the disease progression and treatment efficacy monitoring of cognitive impairment can be achieved.

The above description is only a preferred embodiment of the present invention and does not limit the present invention in any form. Any simple modifications, equivalent changes, and modifications made to the above embodiments based on the technical essence of the present invention still fall within the scope of protection of the technical solution of the present invention.

What is claimed is:

1. An imaging method for cognitive brain science based on a metaverse mixed reality technology, comprising the following steps:
(1) modeling a brain tissue based on magnetic resonance imaging (MRI) images: reconstructing each model with a marching cubes (MC) algorithm extracting contour surfaces according to markers of different brain tissues in MRI data, the MC algorithm dividing three-dimensional (3D) volume data into a series of small cubes, and processing each of the series of small cubes one by one;
(2) virtual modeling fiber bundles: obtaining a spatial transformation matrix between the fiber bundles and tissues through physical space matching, and selecting fiber bundle branches that need to be reconstructed based on tissue identification;
(3) optimizing a mesh model: selecting an edge with a lowest collapse cost with an edge collapse simplification algorithm based on a quadratic measurement error method, then merging two endpoints of the edge, and removing a triangle related to the edge; and repeating this process several times to reduce the mesh model to a size meeting requirements; and
(4) converting and generating a complete model: performing surface reduction processing on the stereolithography STL file with digital carving and drawing a ZBrush software to obtain a processed STL file, converting the processed STL file into an object file in a 3D model file format of OBJ, packaging and coloring the object file, integrating a plurality of corresponding STL files into the complete model, and finally exporting a file in a format of SLN.

2. The imaging method for the cognitive brain science based on the metaverse mixed reality technology according to claim 1, wherein in the step (1), dividing the 3D volume data into the series of small cubes, and analyzing vertex states of each of the series of small cubes one by one; generating a binary index to query a predefined lookup table and determine corresponding triangular facets with a relationship between marker values of 8 vertices of each of the series of small cubes and a preset threshold; and constructing a relatively smooth surface on a cube boundary to complete reconstruction of a 3D model by inserting equivalent points on a corresponding edge with a linear interpolation method.

3. The imaging method for the cognitive brain science based on the metaverse mixed reality technology according to claim 2, wherein the step (2) comprises the following sub steps:
(2.1) calculating a direction vector of each point in the fiber bundles, wherein for a predetermined point in the fiber bundles pᵢ, the direction vector dᵢ of the predetermined point pᵢ is:
$${d}_{i} = {p}_{i + 1} - {p}_{i}$$
(2.2) normalizing the direction vector dᵢ of the predetermined point pᵢ and converting the direction vector dᵢ of the predetermined point pᵢ into color information:
$${d}_{i}^{′} = \frac{\vert {d}_{i} \vert}{max ( \vert {d}_{i} \vert ) + ϵ} \cdot 255$$
wherein ϵ is a small constant to prevent zero division, and |dᵢ| is an absolute value of each component of the direction vector dᵢ; and
(2.3) representing each nerve bundle by a series of continuous 3D coordinate points; firstly, calculating a tangent vector T_{i,i+1} of each point on a nerve bundle path, wherein the tangent vector T_{i,i+1} describes a local direction of the nerve bundle path and generates a rotation axis of a cylindrical cross-section; meanwhile, calculating a normal Nᵢ of each point, wherein the normal Nᵢ provides a normal vector for a generated circular cross-section; for each nerve bundle point, generating a cylindrical cross-section around each nerve bundle point, and calculating positions of each vertex generated on the cylindrical cross-section around each nerve bundle point as follows:
$$P ( t ) = {P}_{i} + {r}_{i} \cdot ( cos ( t ) \cdot {N}_{i} + sin ( t ) \cdot {T}_{i , i + 1} )$$
wherein Pᵢ is a coordinate of a current point in the nerve bundle; rᵢ is a pipe radius of the current point and is preset according to characteristics of the nerve bundle; t is an angle parameter on a circumference with a range of [0, 2π] to generate a complete cylindrical cross-section; the complete cylindrical cross-section connects each pair of adjacent points along the nerve bundle path, forming a complete cylinder; the complete cylindrical cross-sections are connected one by one along a direction of the nerve bundle path to construct a continuous pipe; connection points of cylindrical segments are calculated with the linear interpolation method and smooth transition of normal vectors to ensure a smooth transition on a pipe surface.

4. The imaging method for the cognitive brain science based on the metaverse mixed reality technology according to claim 3, wherein the step (3) further comprises: performing Laplace smoothing on the mesh model, and moving a coordinate of each vertex according to a mean value of coordinates of surrounding vertices of each vertex.

5. The imaging method for the cognitive brain science based on the metaverse mixed reality technology according to claim 4, wherein in the step (4), after obtaining the file in the format of SLN, opening the file in the format of SLN with Visual Studio 2019 and generating a program package to obtain a file in a format of MSIX.

6. The imaging method for the cognitive brain science based on the metaverse mixed reality technology according to claim 5, wherein in the step (4), importing the file in the format of SLN into a 3D reconstruction workstation to adjust color and transparency.

7. The imaging method for the cognitive brain science based on the metaverse mixed reality technology according to claim 6, further comprising step (5), wherein in the step (5), uploading the file in the format of MSIX to cloud storage after generating the file in the format of MSIX.

8. The imaging method for the cognitive brain science based on the metaverse mixed reality technology according to claim 7, further comprising step (6), wherein in the step (6), exporting data as a file in a format of V3D and generating a corresponding quick response (QR) code with the file in the format of V3D.

9. An imaging means for cognitive brain science based on a metaverse mixed reality technology, comprising:
\textcircled{1} a model of modeling brain tissues based on MRI images, wherein the model of modeling the brain tissues is configured to reconstruct each model with an MC algorithm extracting contour surfaces according to markers of different brain tissues in MRI data, the MC algorithm dividing 3D volume data into a series of small cubes, and processing each of the series of small cubes one by one;
\textcircled{2} a model of virtual modeling fiber bundles, wherein the model of virtual modeling the fiber bundles is configured to obtain a spatial transformation matrix between the fiber bundles and tissues through physical space matching, and select fiber bundle branches that need to be reconstructed based on tissue identification;
\textcircled{3} a model of optimizing a mesh model, wherein the model of optimizing the mesh model is configured to select an edge with a lowest collapse cost with an edge collapse simplification algorithm based on a quadratic measurement error method, then merge two endpoints of the edge, and remove a triangle related to the edge; and repeat this process several times to reduce the mesh model to a size meeting requirements; and
\textcircled{4} a module of converting and generating a complete model, wherein the module of converting and generating the complete model is configured to perform surface reduction processing on a stereolithography STL file with a ZBrush software to obtain a processed STL file, convert the processed STL file into an object file in a 3D model file format of OBJ, package and color the object file, integrate a plurality of corresponding STL files into the complete model, and finally export a file in a format of SLN.

10. The imaging means for the cognitive brain science based on the metaverse mixed reality technology according to claim 9, further comprising a module of uploading, wherein the module of uploading is configured to upload a file in a format of MSIX to cloud storage after generating the file in the format of MSIX.

11. The imaging means for the cognitive brain science based on the metaverse mixed reality technology according to claim 10, further comprising a module of QR code, wherein the module of QR code is configured to export data as a file in a format of V3D, and generate a corresponding QR code with the file in the format of V3D or directly input the file in the format of V3D and the corresponding QR code into a mixed reality glasses HoloLens 2.
